(19) United States
(12) Patent Application Publication
(10) Pub. No.: US 2006/0210486 A1
(43) Pub. Date: Sep. 21, 2006

(54) Aerosol antiperspirant with polyamide

(75) Inventors: Anthony Esposito, Roselle, NJ (US); Aixing Fan, Bridgewater, NJ (US)

Correspondence Address: COLGATE-PALMOLIVE COMPANY, 909 RIVER ROAD, PISCATAWAY, NJ, 08855, US

(73) Assignee: COLGATE-PALMOLIVE COMPANY, New York, NY (US)

(21) Appl. No.: 10/547,450

(22) PCT Filed: Mar. 2, 2004
(86) PCT No.: PCT/US04/06164

§ 371 Date: Apr. 17, 2006

Related U.S. Application Data
(60) Provisional application No. 60/451,282, filed on Mar. 4, 2003.

(51) Int. Cl. A61K 9/00

(52) U.S. Cl. 424/47; 424/65

(57) ABSTRACT

An aerosol antiperspirant/deodorant comprising: (a) a solvent system for the gellant in an amount of up to 36% of the total formula; (b) from 0.1-1.5% by weight based on the total weight of the composition of at least one siliconized polyamide as a gellant; (c) an antiperspirant active used in an amount to have a deodorant and/or antiperspirant effect; and (d) a propellant gas.

### FIELD OF THE INVENTION

This invention relates to aerosol compositions that have improved properties. These improvements include easier manufacturing control and reproducibility. The manufactured products help alleviate hard packing of the antiperspirant active to the bottom of the can. This ensures that the active ingredient is dispensed evenly when the product is used.

### BACKGROUND OF THE INVENTION

Selected polyamides as gellants have been disclosed in previous cases owned by the same owner as this invention. It has now been found that this material can advantageously be used in aerosol compositions under certain conditions.

### BRIEF SUMMARY OF THE INVENTION

The invention comprises an aerosol product comprising:

(a) about 2-20 weight % cyclomethicone (especially 5%) (particularly D5, D6 or mixtures of D5 and D6);

(b) about 1-8 weight % isopropyl palmitate (especially 2.2%);

(c) about 0.5-3.5 weight % mineral oil (especially 0.7%);

(d) about 0.3-4 weight % isostearyl alcohol (especially 0.6%);

(e) about 0.1-1.5 weight % Nylon 611/dimethicone copolymer (especially 0.3%);

(f) about 5-25 weight % of a solid antiperspirant active (especially 10%);

(g) optionally about 0.3-2.0 weight % of a perfume oil (especially 1.0%); and

(h) about 37-90 weight % of a propellant gas (especially 83%);

wherein the amounts are based on the total weight of the composition.

### DETAILED DESCRIPTION OF THE INVENTION

The aerosol formulation is made from:

(a) a solvent system for the gellant in an amount of up to 36% of the total formula, wherein the solvent system is compatible with the siliconized polyamide and the solvent system comprises one or more members is selected from the group consisting of:

(1) from 1.5-16% by weight based on the total weight of the composition of at least one non-silicone organic material selected from the group consisting of C12-36 esters (for example, tridecyl neopentanoate, ethyl oleate, dioctyl carbonate, isopropyl palmitate, octyl methoxycinnamate); extra light to heavy white mineral oils with viscosity ranging from 6.5-110 centistokes at 40 degrees C.; guerbet alcohols having 8-30 carbons; fatty alcohols having 8-30 carbons; ethoxylated and propoxylated alcohols having 3-30 carbons (for example, PPG-14 butyl ether, and PPG-3 myristyl ether); alkyl ethers having 12-36 carbons (for example, dioctyl ether); C12-18 alkyl benzoate and benzoate ester derivatives (for example, C12-15 alkyl benzoate, isostearyl benzoate and octyl dodecyl benzoate, octyl salicylate); and paraffins having a distillation temperature in the range of 372-426 degrees C.; isoparaffins having a distillation temperature in the range of 178-207 degrees C.; alkyl carbonates (for example, dioctyl carbonate);

(2) from 2-20% by weight based on the total weight of the composition of a volatile silicone selected from the group consisting of cyclomethicone and low viscosity dimethicones (for example, Dow Corning 200 Fluid/2 centistokes or less from Dow Corning, Midland, Mich.); and

(3) from 0-10% organo-silicones such as phenyl trimethicone. This solvent system consists of one or more of the above listed ingredients. The solvent system also allows the compositions of the invention to be processed at lower temperatures (for example, temperatures in the range of about 60 to 90 degrees C. or lower). This is important in reducing the evaporation of volatile materials from the composition during manufacturing and processing. It should also be noted that many of the solvents described have emollient characteristics in the overall formula.
(b) from 0.1-1.5% by weight based on the total weight of the composition of at least one siliconized polyamide of Formula IIIA as a gellant: 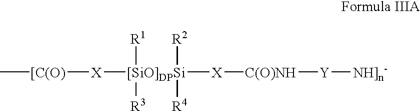
where:

(1) DP is a number in the range of 5-30, particularly 12-18 (especially 15);

(2) n is a number selected from the group consisting of 1-500 (especially 52);

(3) X is a linear or branched chain alkylene having 1-30 carbons (especially 10);

(4) Y is selected from the group consisting of linear and branched chain alkylenes having 1-40 carbons (especially 6), wherein:
(a1) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C1-C3 alkyls; and
(b1) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y=Z² where 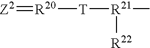
   wherein each of R²⁰, R²¹ are independently selected from the group consisting of linear and branched C1-C10 alkylenes; R²² is selected from the group consisting of linear and branched C1-C10 alkanes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl, especially methyl and ethyl and most especially methyl; and

(5) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl (with more particular values for R¹-R¹ being selected from methyl and ethyl and especially methyl);

wherein the polyamide of Formula IIIA has:

(i) a silicone portion in the acid side of the polyamide;
(ii) an average molecular weight of at least 30,000 daltons with at least 95% of the polyamide having a molecular weight greater than 4,000 daltons; and
(iii) a polydispersity of less than 20;
   (The polyamide material is supplied by Dow Corning Corp., Midland Mich., as Dow Corning® 2-8178 Gellant. The INCI Name is Nylon-611/Dimethicone copolymer, but the commercial version of the product conveniently used here has abut 10 weight % PPG-3 myristal ether. Thus, the PPG-3 myristal ether can also be in the final composition if this commercial material is used. For the final composition, the amount of PPG myristal ether can be in the range of about 0.01-0.15 weight %.)
   (c) an antiperspirant active used in an amount to have a deodorant and/or antiperspirant effect (for example, 5-25 weight % based on the entire weight of the composition) for example Reach® 103 an activated antiperspirant or Reheis Microdry® ACH a non-activated aluminum chlorohydrate (“ACH”) (from Reheis Incorporated, Berkeley Heights, N.J.), wherein the amounts are in percent by weight based on the total weight of the composition. It is preferred to add the antiperspirant active as a dry powder to obtain a product with better efficacy and aesthetics. Solutions of the antiperspirant salt can also be used. These solutions can be solutions in water, glycols, alcohols and mixtures of the above. The solution concentrations can be from 5-50% ACH in the solvent.
   (d) the propellant gas can be chosen from the group singly or in combinations from the following;
(1) compressed gas propellants selected from the group consisting of the industry standard inert gases carbon dioxide, nitrous oxide, and nitrogen;
(2) liquefied gas propellants selected from the groups consisting of:
   (a) hydrocarbons, for example liquefied petroleum gases such as propane, isobutane, n-butane, isopentane and n-pentane;
   (b) fluorocarbons, for example 1,1Difluoroethane (152a) and 1,1,1,2 Tetrafluoroethane (134a);
   (c) ethers, for example, dimethyl ether; and
   (d) mixtures of the foregoing (a)-(c).

Optional ingredients may also be added to the composition of the invention. These optional ingredients include additional emollients (0-20%), silicone gums (0-20%), elastomers (0-20%), silicone resins (0-20%), colorants (0-1%), fragrances (0-3%), antimicrobials (0-2%), surfactants (0-10%), and inert particulates (0-30%) to achieve better structural integrity, stability or aesthetics.

A variety of aluminum salts can be used in the invention such as is known in the art. While several specific aluminum salts have been described, it is currently believed than any aluminum salt known to be useful in antiperspirant/deodorant products may be used with the invention. While the use of antiperspirant salts containing zirconium (for example, tri and tetra salts of aluminum and zirconium with glycine) are currently not permitted to be used in aerosol products in many geographies including the United States, there is no technical reason why such salts cannot be used in the invention.

A comprehensive list of both aluminum and aluminum/zirconium salts include the following. These include conventional aluminum and aluminum/zirconium salts, as well as aluminum/zirconium salts complexed with a neutral amino acid such as glycine, as known in the art. See each of European Patent Application Number. 512,770 A1 and PCT case WO 92/19221. Suitable materials include (but are not limited to) aluminum chlorides (various types including, for example, anhydrous form, hydrated form, etc.), basic aluminum chlorides, basic aluminum chlorides combined with zirconyl oxychlorides and hydroxychlorides, and organic complexes of each of basic aluminum chlorides with or without zirconyl oxychlorides and hydroxychlorides and mixtures of any of the foregoing. These include, by way of example (and not of a limiting nature), aluminum chlorohydrate, aluminum chloride, aluminum sesquichlorohydrate, aluminum chlorohydrol-propylene glycol complex, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum dichlorohydrate, aluminum chlorohydrex PG, aluminum chlorohydrex PEG, aluminum dichlorohydrex PG, aluminum dichlorohydrex PEG, aluminum zirconium trichlorohydrex gly propylene glycol complex, aluminum zirconium trichlorohydrex gly dipropylene glycol complex, aluminum zirconium tetrachlorohydrex gly propylene glycol complex, aluminum zirconium tetrachlorohydrex gly dipropylene glycol complex, and mixtures of any of the foregoing.

If aluminum/zirconium salts are used, particular antiperspirant actives that can be incorporated in the compositions of the present invention include the enhanced efficacy aluminum salts and the enhanced efficacy aluminum/zirconium salt-glycine materials, having enhanced efficacy due to improved molecular distribution, known in the art and discussed, for example, in PCT No. WO92/19221. Particular actives include Westchlor A2Z 4105 aluminum zirconium tetrachlorohydrex gly propylene glycol complex, (from Westwood Chemical Corporation, Middletown, N.Y.); Westchlor ZR 35B aluminum zirconium tetrachlorhydrex gly, and Rezal 36 GP and AZP 902 aluminum zirconium tetrachlorhydrex gly both from Reheis, Berkeley Heights, N.J. as well as Rezal AZZ 908 from Reheis. In general, the metal:chloride mole ratio is in the range of 2.1-0.9:1 for such salts.

Another particular group of actives of special interest because they form low RI solutions include: Westchlor Zr 35BX3 (30-35% actives in water) from Westwood Chemical Company, Middletown, N.Y.; Rezal 36G (46% in water) from Reheis Inc., Berkeley Heights, N.J.; Summit AZG-368 (28-32% in water) from Summit Research Labs, Huguenot, N.Y.; Reach 301 (39% in water) from Reheis Inc.; and aluminum chloride (28% in water) which may be obtained from several sources. In general, the metal:chloride mole ratio is approximately 1.4:1 for such salts.

If an aluminum zirconium salt is used, one particular type of salt of interest is an aluminum zirconium tetra salt with glycine is used wherein aluminum zirconium tetrachlorohydrex glycine salt having a metal to chloride ratio in the range of 0.9-1.2:1 (especially in the range of 0.9-1.1:1 and, more particularly in the range of 0.9-1.0:1); and a glycine:zirconium mole ratio greater than 1.3:1, particularly greater than 1.4:1.

For commercial purposes, currently the particular embodiments of interest contain only aluminum salts and do not contain zirconium.

Other embodiments are free of silicone gums, elastomers, silicone resins, colorants, fragrances, antimicrobials, surfactants, and inert particulates.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997).

### Example 1

### Aerosol Product

Base Formula—An aerosol base is produced by combining 23.4 g cyclopentasiloxane, 14.0 g isopropyl palmitate, 6.8 g mineral oil, 3.8 g isostearyl alcohol, 2 g nylon 611/dimethicone copolymer (also referred to herein as “selected polyamide”) together with stirring and heating to a temperature of about 90 degrees C. until all of the selected polyamide is dissolved. The mixture is cooled to room temperature. Next, 50 g of antiperspirant active are added and dispersed into the resultant gel. A thick antiperspirant base with a viscosity in the range of 3,500-12,000 centipoise as determined by using a Brookfield RTV viscometer. The measurement is taken using Spindle D at 10 RPM with the Helipath attachment.

Filling the Aerosol Cans—The amount of base formula used depends on the final concentration of aluminum active salt required for the final product. The current formula requires 16 g of the aerosol base and 1 g of perfume oil. These ingredients are placed in a conventional 150 ml tin plated, three piece steel aerosol can (obtained from CCL, Harrisonburg, Va.). A steel plated aerosol valve (obtained from Seaquist Perfect, Carey Ill.) is crimped to the top of the can using a Single Head Pneumatic Aerosol Valve Crimper (from Nalbach Engineering Inc., Country Side Ill.). The can is then charged with 83 g of propellant A-46, a mixture of 21.91% propane, 46.84% n-butane, and 30.84% isobutane, using an aerosol burette. An actuator and a hood are than placed over the valve and the product is ready for use.

What is claimed is:

1. An aerosol antiperspirant/deodorant product comprising:
(a) a solvent system for the gellant in an amount of up to 36% of the total formula, wherein the solvent system is compatible with a siliconized polyamide of Formula IIIA and comprises one or more members is selected from the group consisting of:
(1) from 1.5-16% by weight based on the total weight of the composition of at least one non-silicone organic material selected from the group consisting of C12-36 esters; extra light to heavy white mineral oils with viscosity ranging from 6.5-110 centistokes at 40 degrees C.; guerbet alcohols having 8-30 carbons; fatty alcohols having 8-30 carbons; ethoxylated and propoxylated alcohols having 3-30 carbons; alkyl ethers having 12-36 carbons; C12-18 alkyl benzoate and benzoate ester derivatives; paraffins having a distillation temperature in the range of 372-426 degrees C.; isoparaffins having a distillation temperature in the range of 178-207 degrees C.; and alkyl carbonates;
(2) from 2-20% by weight based on the total weight of the composition of a volatile silicone selected from the group consisting of cyclomethicone and low viscosity dimethicones; and
(3) from 0-10% organo-silicones;
(b) from 0.1-1.5% by weight based on the total weight of the composition of at least one siliconized polyamide of Formula IIIA as a gellant: 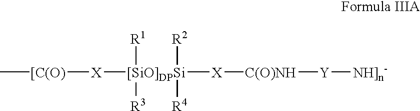
where:
(1) DP is a number in the range of 5-30;
(2) n is a number selected from the group consisting of 1-500;
(3) X is a linear or branched chain alkylene having 1-30 carbons;
(4) Y is selected from the group consisting of linear and branched chain alkylenes having 1-40 carbons, wherein:
(a1) the alkylene group may optionally and additionally contain in the alkylene portion at least one of the members of a group consisting of (i) 1-3 amide linkages; (ii) C5 or C6 cycloalkane as a cycloalkylene linkage; and (iii) phenylene optionally substituted by 1-3 members selected independently from the group consisting of C1-C3 alkyls; and
(b1) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y=Z² where 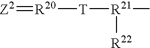
wherein each of R²⁰, R²¹ are independently selected from the group consisting of linear and branched C1-C10 alkylenes; R²² is selected from the group consisting of linear and branched C1-C10 alkanes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl; and
(5) each of R1-R4 is independently selected from the group consisting of methyl, ethyl, propyl, isopropyl, a siloxane chain, and phenyl, wherein the phenyl may optionally be substituted by 1-3 members from the group consisting of methyl and ethyl;
wherein the polyamide of Formula IIIA has:
(i) a silicone portion in the acid side of the polyamide;
(ii) an average molecular weight of at least 30,000 daltons with at least 95% of the polyamide having a molecular weight greater than 4,000 daltons; and
(iii) a polydispersity of less than 20;
(c) an antiperspirant active used in an amount to have a deodorant and/or antiperspirant effect; and
(d propellant gas selected from the group consisting of:
(1) compressed gas propellants selected from the group consisting of inert gases, carbon dioxide, nitrous oxide, and nitrogen;
(2) liquefied gas propellants selected from the groups consisting of:
(i) hydrocarbons selected from the group consisting of liquefied petroleum gases;
(ii) fluorocarbons selected from the group consisting of 1,1 difluoroethane (152a) and 1,1,1,2 tetrafluoroethane (134a);
(iii) ethers; and
(iv) mixtures of the foregoing (i)-(iii).

2. An aerosol antiperspirant/deodorant product comprising:
(a) about 2-20 weight % cyclomethicone;
(b) about 1-8 weight % isopropyl palmitate;
(c) about 0.5-3.5 weight % mineral oil;
(d) about 0.3-4 weight % isostearyl alcohol;
(e) about 0.1-1.5 weight % Nylon 611/dimethicone copolymer;
(f) about 5-25 weight % of a solid antiperspirant active;
(g) optionally about 0.3-2.0 weight % of a perfume oil; and
(h) about 37-90 weight % of a propellant gas;
wherein the amounts are based on the total weight of the composition.

3. An aerosol antiperspirant/deodorant product according to claim 2 wherein the cyclomethicone is D5, D6 or mixtures of D5 and D6 cyclomethicones.

4. An aerosol antiperspirant/deodorant product according to claim 2 comprising:
(a) about 5 weight % cyclomethicone;
(b) about 2.2 weight % isopropyl palmitate;
(c) about 0.7 weight % mineral oil;
(d) about 0.6 weight % isostearyl alcohol;
(e) about 0.3 weight % Nylon 611/dimethicone copolymer;
(f) about 5-25 weight % of a solid antiperspirant active (especially 10%);
(g) optionally about 0.3-2.0 weight % of a perfume oil (especially 1.0%); and
(h) about 37-90 weight % of a propellant gas (especially 83%);
wherein the amounts are based on the total weight of the composition.

5. An aerosol antiperspirant/deodorant product according to claim 2 wherein the propellant gas is one or more gasses selected from the group consisting of:
(a) compressed gas propellants selected from the group consisting of the industry standard inert gases carbon dioxide, nitrous oxide, and nitrogen; and
(b) liquefied gas propellants selected from the groups consisting of:
(1) hydrocarbons which are liquefied petroleum gases selected from the group consisting of propane, isobutene, n-butane, is pentane and n-pentane;
(2) fluorocarbons selected from the group consisting of 1,1-difluoroethane (152a) and 1,1,1,2-tetrafluoroethane (134a);
(3) diethyl ether; and
(4) mixtures of any of the foregoing.

6. An aerosol antiperspirant/deodorant product according to claim 1 further comprising one or more additional ingredients selected from the group consisting of: emollients, silicone gums, elastomers, silicone resins, colorants, fragrances, antimicrobials, surfactants, and inert particulates.

7. An aerosol antiperspirant/deodorant product according to claim 2 further comprising one or more additional ingredients selected from the group consisting of: emollients, silicone gums, elastomers, silicone resins, colorants, fragrances, antimicrobials, surfactants, and inert particulates.

8. An aerosol antiperspirant/deodorant product according to claim 1 which is free of silicone gums, elastomers, silicone resins, colorants, fragrances, antimicrobials, surfactants, and inert particulates.

9. An aerosol antiperspirant/deodorant product according to claim 1 which is free of silicone gums, elastomers, silicone resins, colorants, fragrances, antimicrobials, surfactants, and inert particulates.
